(19) United States
(12) Patent Application Publication
(10) Pub. No.: US 2022/0111012 A1
(43) Pub. Date: Apr. 14, 2022

(54) METHODS OF PROMOTING REMYELINATION

(71) Applicant: Mallinckrodt ARD IP Unlimited Company, Dublin (IE)

(72) Inventors: Dale Wright, Hazelwood, MO (US); Rick Fitch, Hazelwood, MO (US)

(21) Appl. No.: 17/420,622

(22) PCT Filed: Jan. 7, 2020
(86) PCT No.: PCT/US2020/012561

§ 371 Date: Jul. 2, 2021

Related U.S. Application Data
(60) Provisional application No. 62/789,016, filed on Jan. 7, 2019.
(60) Provisional application No. 62/852,097, filed on May 23, 2019.

(51) Int. Cl. A61K 38/35; A61K 9/00; A61P 25/00

(52) U.S. Cl. CPC A61K 38/35; A61P 25/00; A61K 9/0019

(57) ABSTRACT

The present disclosure is directed to methods of increasing remyelination in subjects in need thereof.

### CLAIM OF PRIORITY

This application claims priority to U.S. Provisional Application No. 62/789,016; filed Jan. 7, 2019 and to U.S. Provisional Application No. 62/852,097; filed May 23, 2019, the entire contents of which are hereby incorporated by reference.

### FIELD OF THE DISCLOSURE

The present disclosure is directed to methods of increasing remyelination in subjects in need thereof.

### BACKGROUND OF THE DISCLOSURE

Myelination is a complex process that is regulated on several levels. In diseases or disorders related to myelination, at least two distinct processes are involved. One is the loss of myelination (e.g. demyelination), and another is the inhibition of remyelination. Compounds or treatments that slow or inhibit demyelination do not necessarily promote remyelination, and vice versa. For instance, steroids have been shown to slow demyelination, but inhibit remyelination. As remyelination is critical for the restoration of neuronal health and function, there is a need in the art for treatments that not only inhibit demyelination, but also promote remyelination.

### SUMMARY OF THE DISCLOSURE

One aspect of the present disclosure encompasses a method of increasing remyelination in a subject in need thereof. The method comprises administering a repository corticotropin injection (RCI) to the subject in need of remyelination, and subsequently performing a diagnostic test for myelination.

Another aspect of the present disclosure encompasses a method of increasing remyelination in a subject in need thereof, where the method comprises administering repository corticotropin injection to the subject, wherein the subject is not contemporaneously administered steroids.

Yet another aspect of the present disclosure encompasses a method of increasing remyelination in a subject in need thereof, where the method comprises administering a repository corticotropin injection to the subject in an amount sufficient to show increased remyelination in a subsequent diagnostic test.

Other aspects and iterations of the present disclosure are detailed below.

### BRIEF DESCRIPTION OF THE FIGURES

FIG. 1 depicts micrographs illustrating PPD staining of myelinated axons in the corpus callosum (white matter). Black color is positive staining for PPD; see arrows for stained areas of interest.

FIG. 2 depicts a graph showing the density of myelinated axons in the corpus callosum (white matter). *p<0.05; †p<0.0001

FIG. 3 depicts a graph showing PDGFRα immunohistochemistry staining within the corpus callosum.

### DETAILED DESCRIPTION

The present disclosure encompasses methods of increasing remyelination. Generally speaking, the methods comprise administering to a subject in need of remyelination an ACTH composition. In preferred embodiments, the ACTH composition is a repository corticotropin injection.

### I. Compositions of the Disclosure

One aspect of the present disclosure encompasses an ACTH composition. Suitable ACTH compositions are described in detail below.

### (a) ACTH

ACTH is a 39 amino acid peptide hormone that is secreted by the pituitary gland and is a part of the hypothalamus-pituitary-adrenal (HPA) axis that maintains the stress response and homeostasis in the body. Physiologically, the principal effects of ACTH are stimulation of the adrenal cortex with subsequent increased production of glucocorticosteroids and/or cortisol from the adrenal cortex. ACTH levels are tightly regulated in the body via a negative feedback loop wherein glucocorticosteroids suppress the release of corticotropin release hormone (CRH) from the pituitary and CRH-mediated release of ACTH. In some instances, cortisol helps restore homeostasis after stress. In some instances, changed patterns of serum cortisol levels are observed in connection with abnormal ACTH levels. In some instances, prolonged ACTH-mediated secretion of abnormal levels of cortisol (e.g., higher or lower levels of cortisol compared to cortisol levels in normal individuals) has detrimental effects. Thus, any perturbation in the levels of ACTH has profound physiological implications.

ACTH is synthesized from a precursor polypeptide pre-pro-opiomelanocortin (pre-POMC). The removal of the signal peptide during translation produces a 267 amino acid polypeptide POMC. POMC undergoes a series of post-translational modifications to yield various polypeptide fragments including and not limited to ACTH, β-lipotropin, γ-lipotropin, α, β, γ-Melanocyte Stimulating Hormone (MSH) and β-endorphin. POMC, ACTH and β-lipotropin are also secreted from the pituitary gland in response to the hormone corticotropin-releasing hormone (CRH). The first 13 amino acids of ACTH₁₋₃₉ are cleaved to form α-melanocyte-stimulating hormone (α-MSH).

In some instances, an abnormality in ACTH levels is associated with inflammation (e.g., increased release of pro-inflammatory cytokines). In some instances, an abnormality in ACTH levels is associated with reduced VEGF secretion. In some instances, reduced VEGF secretion is associated with reduced growth of new blood vessels and inadequate oxygen supply to tissues (e.g., neurons and/or muscles).

In some embodiments of the use or methods described herein, the ACTH peptide is a ACTH₁₋₃₉ peptide having the formula:

```
H-Ser-Tyr-Ser-Met-Glu-His-Phe-Arg-Trp-Gly-
        1 2 3 4 5 6 7 8 9 10
Lys-Pro-Val-Gly-Lys-Lys-Arg-Arg-Pro-Val-
  11 12 13 14 15 16 17 18 19 20
Lys-Val-Tyr-Pro-Asp-Gly-Ala-Glu-Asp-Gln-
  21 22 23 24 25 26 27 28 29 30
Leu-Ala-Glu-Ala-Phe-Pro-Leu-Glu-Phe-OH
  31 32 33 34 35 36 37 38 39
```

or a fragment thereof, a variant thereof or any combination thereof.

The term “ACTH”, in some embodiments, includes corticotropin, adrenocorticotropic hormone, Tetracosactide or the like. In some embodiments, the term ACTH includes a 39 amino acid peptide hormone secreted by the anterior pituitary gland. In other embodiments the term “ACTH” also includes any ACTH peptide, any ACTH fragment, or any ACTH preparation as described herein. The term ACTH includes, in some embodiments, ACTH from any source including human ACTH, mouse ACTH, rat ACTH, porcine ACTH, sheep ACTH, bovine ACTH, rabbit ACTH or any other source of ACTH. In further embodiments, the term ACTH includes humanized and/or recombinant forms of ACTH and synthetic forms of ACTH.

The term “ACTH peptide” refers to a ACTH₁₋₃₉ peptide. The term “ACTH peptide homolog” includes ACTH peptide or peptide fragments or ACTH-like compounds with about 40%, about 45%, about 50%, about 55%, about 60%, about 65%, about 70%, about 75%, about 80%, about 85%, about 90%, or about 95% sequence identity with ACTH₁₋₃₉.

As used herein the term “variant” may mean a peptide or polypeptide that differs in amino acid sequence by the insertion, deletion, or conservative substitution of amino acids, but retain at least one biological activity. A variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

The term “ACTH aggregate” refers to a physical grouping of peptides which may comprise ACTH peptide, or fragments, analogs or homologs thereof. Such an aggregate may comprise hydrogen-bonded molecules and/or molecules held by bridging interactions via, for example, a salt bridge, a metal ion, and the like.

The term “ACTH complex” refers to ACTH or fragments or variants thereof that are optionally complexed with other proteins (e.g., Bovine Serum Albumin), or metal ions, or charged polymers (e.g., polylysine), or fragments, homologs or analogs of ACTH, or any other suitable complexes that retain the functional characteristics of ACTH or ACTH fragments or analogs thereof and/or allow for formulation of ACTH or ACTH fragments or analogs thereof into suitable dosage forms.

In some embodiments, ACTH is an ACTH preparation. As used herein, “ACTH preparation” refers to a mixture containing ACTH peptide and/or other peptide fragments and/or other proteins and/or other substances that together form a composition that is suitable for any methods and/or dosing regimen described herein. In some of such embodiments, ACTH is obtained from a homogenized pituitary extract of an appropriate animal (e.g., pituitary extract of a pig). Any suitable method may be used to obtain a homogenized pituitary extract. In some embodiments, a homogenized pituitary extract includes ACTH peptide and/or other peptide fragments and/or other proteins and/or other substances that are contemplated as being part of the ACTH preparation that is compatible with any method described herein.

In one aspect, an ACTH composition refers to a repository corticotropin injection. For instance, in one embodiment, an ACTH composition that is a repository corticotropin injection is Acthar gel (also referred to as H.P. Acthar gel). Acthar Gel is a naturally sourced complex mixture of purified adrenocorticotropic hormone analogs and other pituitary peptides. The Acthar Gel manufacturing process converts the initial porcine pituitary extract with low ACTH content into a mixture having modified porcine ACTH and other related peptide analogs solubilized in gelatin. A major component in the formulated complex mixture is N-25 deamidated porcine ACTH (1-39). Acthar Gel is supplied as a sterile preparation in 16% gelatin to provide a prolonged release after intramuscular or subcutaneous injection. Acthar Gel also contains 0.5% phenol, not more than 0.1% cysteine (added), sodium hydroxide and/or acetic acid to adjust pH and water for injection.

In particular embodiments, an ACTH composition of the present disclosure refers to a composition comprising N-25 deamidated porcine ACTH (1-39).

In some embodiments, Acthar gel may be administered intramuscularly and a daily dose of 150 U/m2 (divided into twice daily intramuscular injections of 75 U/m²) may be administered over a 2-week period. The dosing with Acthar gel can be gradually tapered over a 2-week period to avoid adrenal insufficiency. In one exemplary embodiment, a tapering schedule is as follows: 30 U/m² in the morning for 3 days; 15 U/m² in the morning for 3 days; 10 U/m² in the morning for 3 days; and 10 U/m² every other morning for 6-days.

Acthar gel is typically dosed based on body surface area (BSA). For calculation of body surface area, use the following formula:

$$BSA ( {m}^{2} ) = \sqrt{\frac{weight ( kg ) \times height ( cm )}{3600}}$$

In other embodiments, Acthar gel may be administered intramuscularly or subcutaneous at doses of 80-120 units for 2-3 weeks for acute exacerbations. In still other embodiments, the dose of Acthar gel may be 40-80 units given intramuscularly or subcutaneously every 24-72 hours. In some embodiments, dosing is individualized according to the medical condition of each patient. Frequency and dose of the drug can be determined by considering the severity of the disease and the initial response of the patient.

Acthar gel is contraindicated for intravenous administration. Acthar gel is contraindicated where congenital infections are suspected in infants. Administration of live or live attenuated vaccines is contraindicated in patients receiving immunosuppressive doses of Acthar Gel. Acthar gel is contraindicated in patients with scleroderma, osteoporosis, systemic fungal infections, ocular herpes simplex, recent surgery, history of or the presence of a peptic ulcer, congestive heart failure, uncontrolled hypertension, primary adrenocortical insufficiency, adrenocortical hyperfunction or sensitivity to proteins of porcine origin.

The term “ACTH peptide, fragment, or variant” also includes, in certain embodiments, pre-POMC, POMC, β-lipotropin, γ-lipotropin, Melanocyte Stimulating Hormone (α-MSH, μ-MSH, γ-MSH), β-endorphin, or the like, or any other polypeptide fragment that is a post-translational product of the POMC gene. POMC genes for various species are found in the NCBI GenBank including and not limited to human POMC transcript variant 1, mRNA, (NCBI Accession number NM_001035256), human POMC transcript variant 2, mRNA, (NCBI Accession number NM_000939), swine pro-opiomelanocortin, mRNA (NCI Accession number S73519), swine proopiomelanocortin protein (POMC) gene (NCBI Accession number EU184858), rat proopiomelanocortin (POMC) gene (NCBI Accession number K01877), or the like. Other examples of POMC genes include, for example, catfish POMC gene described in Animal Genetics, 2005, 36, 160-190. Melanocortin peptides, including ACTH and alpha, beta and gamma MSH derive from post-translational modification of POMC. A number of melanocortin peptides share an invariant sequence of four amino acids, His-Phe-Arg-Trp, which also correspond to residues 6-9 of ACTH and alpha-MSH. Accordingly, also contemplated within the scope of embodiments presented herein, is the use of amino acid sequences that correspond to alpha MSH, beta MSH or gamma MSH. See Catania et al., Pharmacol. Rev. 2004, 56: 1-29.

The term “ACTH peptide, fragment, or variant” includes, in addition to embodiments described above or below, in certain aspects, synthetic preparations of ACTH that are commercially available including and not limited to Acthar® gel, Synacthen®, Adrenomone®, or the like. Examples of commercially available ACTH peptides that are compatible with the methods described herein include and are not limited to Adrenocorticotropic Hormone (ACTH) (1-10) (human), Adrenocorticotropic Hormone (ACTH) (1-13) (human), Adrenocorticotropic Hormone (ACTH) (1-16) (human), Adrenocorticotropic Hormone (ACTH) (1-17) (human), Adrenocorticotropic Hormone (ACTH) (1-24) (human), Adrenocorticotropic Hormone (ACTH) (1-39) (human), Adrenocorticotropic Hormone (ACTH) (1-39) (rat), Adrenocorticotropic Hormone (ACTH) (18-39) (human), Adrenocorticotropic Hormone (ACTH) (4-10) (human), Adrenocorticotropic Hormone (ACTH) (1-4), Adrenocorticotropic Hormone (ACTH) (1-14) or the like available from, for example, GenScript.

The term “prodrug” refers to a precursor molecule that is a derivative of ACTH or ACTH fragments or analogs thereof that is suitable for incorporation in any dosage form described herein. A “prodrug” refers to a precursor compound that is converted into active compound in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, prodrugs facilitate transmittal across a cell membrane where water solubility is detrimental to mobility but which then is metabolically hydrolyzed to the carboxylic acid, the active entity, once inside the cell where water-solubility is beneficial. As non-limiting examples, a prodrug of ACTH or fragment of analog thereof is metabolically stable and is not degraded in the stomach.

Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or less labile and/or confers solubility or some other property to the drug. Once the chemical group has been cleaved and/or modified from the prodrug the active drug is generated. In some embodiments, a prodrug of ACTH or fragment or analog thereof is an alkyl ester of the parent compound such as, for example, methyl ester, ethyl ester, n-propyl ester, iso-propyl ester, n-butyl ester, sec-butyl ester, tert-butyl ester or any other ester.

### (b) Pharmaceutical Compositions

The present disclosure also provides pharmaceutical compositions. The pharmaceutical composition comprises an ACTH peptide, fragment, and variant, or any combination thereof, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the invention may optionally comprise one or more additional drug or therapeutically active agent in addition to the ACTH peptide, fragment, and variant, or any combination thereof. Thus, in addition to the therapies described herein, one may also provide to the subject other therapies known to be efficacious for treatment of the disease, disorder, or condition. In some embodiments, the additional drug or therapeutically active agent induces anti-inflammatory effects.

### (i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

### (ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylam ides, polyvinyloxoazolidone, polyvinylalcohols, C12-C18 fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

### (iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

### (v) pH Modifier

In various embodiments, the excipient may be a pH modifier. By way of non-limiting example, the pH modifying agent may be sodium carbonate, sodium bicarbonate, sodium citrate, citric acid, or phosphoric acid.

### (vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

### (ix) Lubricant

In a further embodiment, the excipient may be a lubricant. Non-limiting examples of suitable lubricants include minerals such as talc or silica; and fats such as vegetable stearin, magnesium stearate, or stearic acid.

### (x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

### (xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

### (c) Administration

### (I) Dosage Forms

A composition of the present disclosure may be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration may include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as ethylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carrier for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising an ACTH peptide, fragment, variant, or any combination thereof, is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the an ACTH peptide, fragment, variant, or any combination thereof, in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, an ACTH peptide, fragment, variant, or any combination thereof may be selectively delivered to a cell by encapsulation in a liposome that fuses with the targeted cell's membrane.

Liposomes may be comprised of a variety of different types of phospholipids having varying hydrocarbon chain lengths. Phospholipids generally comprise two fatty acids linked through glycerol phosphate to one of a variety of polar groups. Suitable phospholipids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tetradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecadienoate (linoleate), all cis-9,12,15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the itaconate, malonate, derivatives thereof, an ACTH peptide, fragment, and variant, or any combination thereof, may be prepared by any known method of preparing liposomes for drug delivery, such as, for example, detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of an ACTH peptide, fragment, variant, or any combination thereof, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The compound of the itaconate, malonate, derivatives thereof, a compound of Formula (I), or a compound of Formula (II) may be encapsulated in a microemulsion by any method generally known in the art.

In yet another embodiment, an ACTH peptide, fragment, variant, or any combination thereof, may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

Generally, a safe and effective amount of an ACTH peptide, fragment, variant, or any combination thereof is, for example, that amount that would cause the desired therapeutic effect in a subject while minimizing undesired side effects. In various embodiments, an effective amount of an ACTH peptide, fragment, variant, or any combination thereof described herein can substantially increase remyelination and as such represents a therapeutic option for treatment of demyelination disorders.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an ACTH peptide, fragment, variant, or any combination thereof can occur as a single event or over a time course of treatment. For example, an ACTH peptide, fragment, variant, or any combination thereof can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for an inflammatory autoimmune disease.

An ACTH peptide, fragment, variant, or any combination thereof can be administered simultaneously or sequentially with another agent, such as an antibiotic, an anti-inflammatory, or another agent. For example, an ACTH peptide, fragment, variant, or any combination thereof can be administered simultaneously with another agent, such as an anti-inflammatory. Simultaneous administration can occur through administration of separate compositions, each containing one or more of an ACTH peptide, fragment, variant, or any combination thereof, an anti-inflammatory, or another agent. Simultaneous administration can occur through administration of one composition containing two or more compositions. An ACTH peptide, fragment, variant, or any combination thereof can be administered sequentially with an antibiotic, an anti-inflammatory, or another agent. For example, an ACTH peptide, fragment, variant, or any combination thereof can be administered before or after administration of an anti-inflammatory, or another agent.

In some embodiments, the first dose is administered upon detection of one or more symptoms of a demyelinating disorder. In some embodiments, the first dose is administered upon detection of loss of myelination. In some embodiments, the one or more subsequent doses are administered every day, every other day, every two days, every three days, every four days, every 5 days, every 6 days, once a week, every two weeks, every three weeks, once a month, every six weeks, every two months, every three months, every four months, every five months, every six months or any combination thereof.

In some embodiments, a first dose of ACTH, fragment, variant, or any combination thereof, is between about 10 IU, about 20 IU, about 30 IU, 40 IU, about 50 IU, about 60 IU, about 70 IU, 80 IU, about 90 IU, about 100 IU, about 110 IU, about 120 IU, about 130 IU, about 140 IU, about 150 IU about 200 IU, about 250 IU, about 300 IU, about 350 IU, about 400 IU, about 450 IU or about 500 IU. In some embodiments, a first dose of ACTH, fragment, or any combination thereof, is between about 10 IU to about 400 IU, between about 10 IU to about 250 IU, between about 10 IU to about 100 IU, between about 10 IU to about 80 IU, between about 10 IU to about 60 IU, or between about 10 IU to about 40 IU. In some embodiments, a first dose of ACTH or fragment, analog, complex or aggregate thereof, or any combination thereof, is between about 10 IU to about 400 IU, between about 20 IU to about 400 IU, between about 40 IU to about 400 IU, between about 40 IU to about 350 IU, between about 40 IU to about 200 IU, between about 40 IU to about 100 IU, between about 40 IU to about 80 IU, or between about 40 IU to about 60 IU. In some embodiments, a first dose of ACTH, fragment, variant, or any combination thereof, is between about 20 IU to about 200 IU, between about 60 IU to about 150 IU, between about 60 IU to about 100 IU, or between about 60 IU to about 80 IU.

In some embodiments, a one or more subsequent dose of ACTH, fragment, variant, or any combination thereof, is between about 10 IU, about 20 IU, about 30 IU, 40 IU, about 50 IU, about 60 IU, about 70 IU, 80 IU, about 90 IU, about 100 IU, about 110 IU, about 120 IU, about 130 IU, about 140 IU, about 150 IU about 200 IU, about 250 IU, about 300 IU, about 350 IU, about 400 IU, about 450 IU or about 500 IU. In some embodiments, a one or more subsequent dose of ACTH, fragment, or any combination thereof, is between about 10 IU to about 400 IU, between about 10 IU to about 250 IU, between about 10 IU to about 100 IU, between about 10 IU to about 80 IU, between about 10 IU to about 60 IU, or between about 10 IU to about 40 IU. In some embodiments, a one or more subsequent dose of ACTH or fragment, analog, complex or aggregate thereof, or any combination thereof, is between about 10 IU to about 400 IU, between about 20 IU to about 400 IU, between about 40 IU to about 400 IU, between about 40 IU to about 350 IU, between about 40 IU to about 200 IU, between about 40 IU to about 100 IU, between about 40 IU to about 80 IU, or between about 40 IU to about 60 IU. In some embodiments, a one or more subsequent dose of ACTH, fragment, variant, or any combination thereof, is between about 20 IU to about 200 IU, between about 60 IU to about 150 IU, between about 60 IU to about 100 IU, or between about 60 IU to about 80 IU.

In some embodiments, the pharmaceutical compositions described herein are in unit dosage forms suitable for single administration of precise dosages. In unit dosage form, the formulation is divided into unit doses containing appropriate quantities of ACTH peptide or fragment, analog, complex or aggregate thereof, or any combination thereof. In some embodiments, the unit dosage is in the form of a package containing discrete quantities of the formulation. Non-limiting examples are packaged tablets or capsules, powders in vials or ampoules, or injectable suspension or solution in ampoules. In some embodiments, aqueous suspension compositions are packaged in single-dose non-reclosable containers. Alternatively, multiple-dose reclosable containers are used. In some of such embodiments, a preservative is optionally included in the composition. By way of example only, formulations for intramuscular injection are presented in unit dosage form, which include, but are not limited to ampoules, or in multi dose containers, with an added preservative.

### II. Methods

One aspect of the present disclosure encompasses methods for increasing remyelination in a subject in need thereof. As used herein, the term “remyelination” refers to the generation of new myelin, whether in white matter or gray matter. Remyelination may be monitored by methods which include direct determination of the state of myelin in the subject, e.g., one can measure white matter mass using magnetic resonance imaging (MRI), measure the thickness of myelin fibers using a magnetic resonance spectroscopy (MRS) brain scan, or any other direct measures known in the art (e.g., Positron-Emission Tomography (PET), Diffusion-Weighted Imaging (DW-I, or DW-MRI), Diffusion Tensor Imaging, Myelography, Magnetization Transfer, etc.). Remyelination may also be indirectly monitored by detecting a reduction in the size or number of inflammatory lesions (i.e., scleroses) present in the patient; monitoring a patient's cerebrospinal fluid (e.g., obtained by a lumbar puncture) for a reduction in the presence or amount of, e.g., (i) abnormal proteins such as tiny fragments of myelin, (ii) elevated levels of or specific types of lymphocytes, and/or (iii) abnormal levels of immunoglobulin (IgG) molecules; monitoring a patient for a positive change in neuropsychology (e.g., the status of various abilities such as memory, arithmetic, attention, judgment and reasoning); and/or monitoring a patient's urine for a decrease in levels of myelin basic protein-like material (MBPLM). Certain tests for color blindness can also be helpful in tracking the treatment of demyelinating disorders on the eyes. Whitaker et al. (1995) Ann Neurol. 38(4):635-632.

Generally speaking, a method of the present disclosure comprises administering an ACTH composition as described in section I above. In preferred embodiments, the methods described herein encompass administering a repository corticotropin injection (RCI) to a subject in need of remyelination.

In one embodiment, the present disclosure encompasses a method of increasing remyelination in a subject in need thereof, where the method comprises administering an ACTH composition to the subject in need of remyelination, and subsequently performing a diagnostic test for myelination. Non-limiting suitable diagnostic tests are described above. In preferred embodiments, the ACTH composition is a repository corticotropin injection. In some embodiments, the diagnostic test is performed at least 1 hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration of an ACTH composition. In each of the above embodiments, the diagnostic test for myelination performed after administration of an ACTH composition to the subject should demonstrate increased myelination compared to the subject before treatment with an ACTH composition.

In another embodiment, the present disclosure encompasses a method of increasing remyelination in a subject in need thereof, where the method comprises administering an ACTH composition to the subject in need of remyelination, wherein the subject is not contemporaneously administered steroids. Steroids are presently known in the art to interfere with remyelination. As a result, administering an ACTH composition of the present invention while administering steroids may inhibit remyelination. In contrast, administering an ACTH composition as described herein in the absence of steroids alleviates the negative impact of the steroids on remyelination and allows for increased remyelination. In preferred embodiments, the subject is administered a repository corticotropin injection.

In another embodiment, the present disclosure encompasses a method of increasing remyelination in a subject in need thereof, where the method comprises administering an ACTH composition to the subject in an amount sufficient to show increased remyelination in a subsequent diagnostic test. Methods of determining such amounts are described in section I above. Non-limiting examples of suitable diagnostic tests are described above. In preferred embodiments, the ACTH composition is repository corticotropin injection.

In further embodiments, the present disclosure encompasses methods of increasing oligodendrocyte progenitor cell proliferation in a subject in need of remyelination. The method comprises administering an ACTH composition to the subject.

A subject in need of remyelination may be a subject diagnosed with a demyelinating disorder of the CNS. For instance, a subject may be diagnosed with a leukodystrophic demyelinating disorder of the CNS, or a subject may be diagnosed with a myelinoclastic demyelinating disorder of the CNS.

In some embodiments, a subject may be diagnosed with a demyelinating disorder of the peripheral nervous system. As used herein, a “demyelinating disorder of the peripheral nervous system” describes a broad class of peripheral neuropathies that are associated with the destruction or removal of myelin, the lipid-rich sheath surrounding and insulating nerve fibers, from nerves. Non-limiting examples of demyelinating peripheral neuropathy diseases may include diabetic peripheral neuropathy, distal sensorimotor neuropathy, or autonomic neuropathies such as reduced motility of the gastrointestinal tract or atony of the urinary bladder. Generally speaking, demyelinating peripheral neuropathies may be genetically acquired, result from a systemic disease, or induced by a toxin or by trauma.

Genetic demyelinating neuropathies (also known as hereditary neuropathies) are one of the most common inherited neurological diseases. Genetic demyelinating neuropathies are divided into four major subcategories: 1) motor and sensory neuropathy, 2) sensory neuropathy, 3) motor neuropathy, and 4) sensory and autonomic neuropathy. Specifically, the demyelinating hereditary neuropathies are often progressive neuropathies with markedly decreased nerve conduction and velocity and chronic segmental demyelination of the peripheral nerve. Gabreels-Festen et al., “Hereditary demyelinating motor and sensory neuropathy,” Brain Pathol. 3(2):135-146 (1993). Examples of general classes of genetic deyelinating neuropathies include but are not limited to diabetic peripheral neuropathy, distal sensorimotor neuropathy, or autonomic neuropathies such as reduced motility of the gastrointestinal tract or atony of the urinary bladder. Examples of hereditary peripheral neuropathies include Charcot-Marie-Tooth disease, Abetalipoproteinemia, Tangier disease, Metachromatic leukodystrophy, Fabry's disease, and Dejerine-Sottas syndrome.

Systemic demyelinating peripheral neuropathies arise as side effects of a systemic illness. Non-limiting examples of peripheral neuropathies associated with systemic disease include post-polio syndrome and AIDS-associated neuropathy. Furthermore, the following non-limiting systemic diseases can have peripheral neuropathy symptoms: cancer, malnutrition, alcoholism, diabetes, AIDS, Lyme disease, Rheumatoid arthritis, chronic kidney failure, autoimmune disorders, hypothyroidism, and viral infections (e.g., hepatitis).

Toxin induced demyelinating peripheral neuropathies are caused by exposure to neurotoxic agents such as pharmaceutical agents, biological agents, and chemical exposure. Examples of toxins that cause peripheral neuropathies include, but are not limited to, chemotherapeutic agents (e.g., vincristine, paclitaxel, cisplatin, methotrexate, or 3′-azido-3′-deoxythymidine), lead, mercury, thallium, organic solvents, pesticides, carbon disulfide, arsenic, acrylamide, diphtheria toxin, alcohol, anti-HIV medications (e.g., didanosine and zalcitabine), anti-tuberculosis medications (e.g., isoniazid and ethambutol), antimicrobial drugs (e.g., dapsone, metronidazole, chloroquine, and chloramphenicol), psychiatric medications (e.g., lithium), radiation, and medications such as amiodarone, aurothioglucose, phenytoin, thalidomide, colchicine, cimetidine, disulfiram hydralazine, and high levels of vitamin B6.

Trauma induced demyelinating peripheral neuropathies, as described above, are caused by bodily shock, injury, or physical trauma.

Accordingly, causes of peripheral neuropathy may range widely, e.g. from diabetic complications; trauma; toxins including, without limitation, drugs and medications, industrial chemicals, and environmental toxins; autoimmune response; nutritional deficiencies; to vascular and metabolic disorders. For example, demyelinating peripheral neuropathies may occur as a result of osteosclerotic myeloma, monoclonal protein-associated peripheral neuropathy, hereditary motor and sensory peripheral neuropathies types 1 and 3, and hereditary susceptibility to pressure palsies.

In certain embodiments, a subject may be diagnosed with a demyelinating disorder of the optic nerve, such as optic neuritis.

In particular embodiments, a subject may be diagnosed with a demyelinating disorder of the CNS that is not multiple sclerosis (MS). Alternatively, a subject may be diagnosed with a demyelinating disorder of the CNS that is not ALS. In another alternative, a subject may be diagnosed with a demyelinating disorder of the CNS that is not ALS or MS.

In some embodiments, at least a 5% (e.g., at least 10%, at least 15%, at least 20%, at least 25%, at least 30%, at least 35%, at least 40%, at least 50%, at least 60%, at least 70%) improvement in one or more symptoms of a neurological disorder characterized by myelin loss or myelin deficiency or other above-described indicia following a remyelination therapy of the disclosure is sufficient to classify the subject as responding to the administration of an ACTH composition.

As used herein the term “therapeutically effective” applied to dose or amount refers to that quantity of a compound or pharmaceutical composition that is sufficient to result in a desired activity upon administration to a subject in need thereof. Within the context of the present invention, the term “therapeutically effective” refers to that quantity of an ACTH composition that is sufficient to delay the manifestation, arrest the progression, relieve or alleviate at least one symptom of a neurological disorder characterized by myelin loss or myelin deficiency. Note that when a combination of active ingredients is administered the effective amount of the combination may or may not include amounts of each ingredient that would have been effective if administered individually.

As used herein, the phrase “neurological disorder characterized by myelin loss or myelin deficiency” or the phrase “demyelination disorder” encompasses any disease associated with the destruction or removal of myelin, the fatty sheath surrounding and insulating nerve fibers, from nerves. Non-limiting examples of disorders characterized by myelin loss or myelin deficiency include, for example, multiple sclerosis (MS) (e.g., Relapsing/Remitting Multiple Sclerosis, Secondary Progressive Multiple Sclerosis, Progressive Relapsing Multiple Sclerosis, Primary Progressive Multiple Sclerosis, and Acute Fulminant Multiple Sclerosis), Central Pontine Myelinolysis, Acute Disseminated Encephalomyelitis, Progressive Multifocal Leukoencephalopathy, Subacute S clerosing Panencephalitis, Post-infectious Encephalomyelitis, Chronic Inflammatory Demyelinating Polyneuropathy, Devic's Disease, Balo's Concentric Sclerosis, the leukodystrophies (e.g., Metachromatic Leukodystrophy, Krabbe disease, Adrenoleukodystrophy, Pelizaeus-Merzbacher disease, Canavan disease, Childhood Ataxia with Central Hypomyelination, Alexander disease, or Refsum disease), optic neuritis, transverse myelitis, cerebral palsy, spinal cord injury, age-associated myelin deficiency, as well as acquired and inherited neuropathies in the peripheral nervous system (e.g., Guillain-Barre Syndrome and Charcot Marie Tooth disease).

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Repository Corticotropin Injection Increases Remyelination

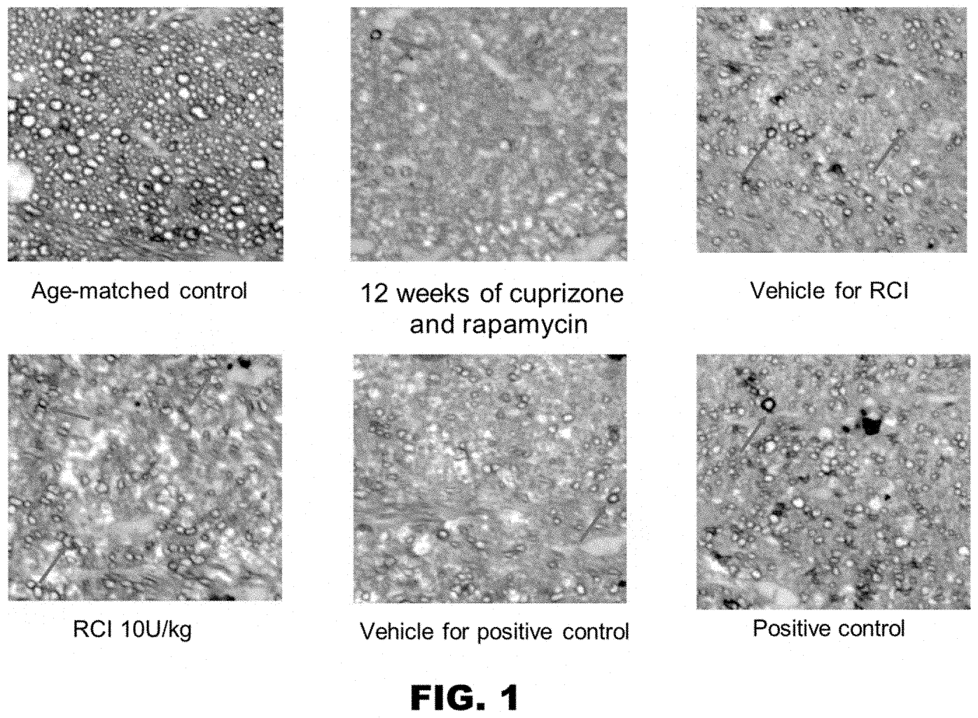

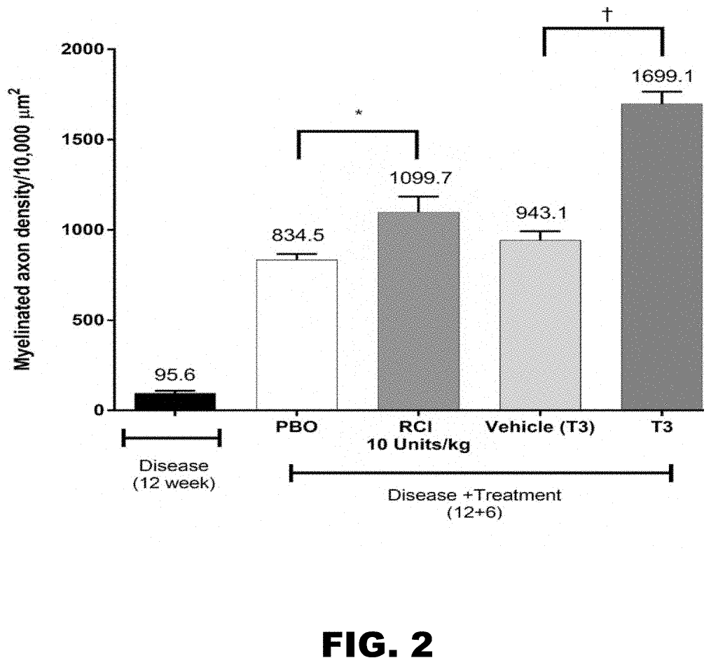

The hypothesis that repository corticotropin injection (RCI) accelerates remyelination in a mouse model of cuprizone demyelination was tested. C57BL/6 mice were fed 0.3% cuprizone co-administered with rapamycin for 12 weeks to induce demyelination and inhibit spontaneous remyelination. After 12 weeks of cuprizone treatment, mice showed severe demyelination of both white and gray matter. Mice were treated for another 6 weeks with RCI, triiodothyronine (T3, positive control), or vehicle (negative control). Remyelination was assayed in gray matter (hippocampus) via quantification of myelin proteolipid protein (PLP) and in white matter (corpus callosum) via p-Phenylenediamine (PPD) staining. RCI significantly increased PLP staining within the hippocampus (gray matter)(see Table 1). Furthermore, RCI treatment increased myelin density in the corpus callosum (white matter) by 31.8% compared to the vehicle (see FIG. 1 and FIG. 2). T3 demonstrated that RCI significantly increased both white and gray matter myelination. Of note—increased remyelination was not observed at higher doses of RCI in this rodent model (data not shown).

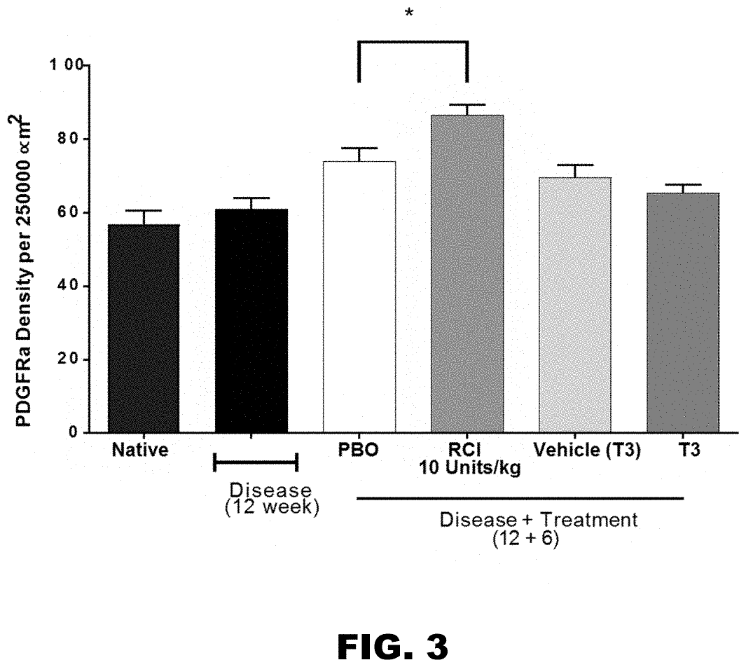

RCI treatment significantly increased immunohistochemical staining for platelet-derived growth factor receptor alpha (PDGFRα), a marker for oligodendrocyte progenitor cells. This suggests that RCI plays a role in oligodendrocyte progenitor cell proliferation to aid in remyelination (FIG. 3).

TABLE 1
Quantification of PLP in the Hippocampus

| Group | Mean PLP (% Area Covered) |
|---|---|
| Age-matched control | 49.0 |
| 12 weeks of cuprizone co-administered | 2.5 |
| with rapamycin | |
| Vehicle Control for RCI | 36.9 |
| RCI 10 U/kg | 38.4* |
| Vehicle for positive control | 37.5 |
| Positive Control | 40.8** |

*p < 0.05 versus vehicle
**p < 0.005 versus vehicle
Abbreviations:
PLP, myeline proteolipid protein;
RCI, repository corticotropin injection

1. A method of increasing remyelination in a subject in need thereof, the method comprising administering a repository corticotropin injection (RCI) to the subject in need of remyelination, and subsequently performing a diagnostic test for myelination.

2. A method of increasing remyelination in a subject in need thereof, the method comprising administering a repository corticotropin injection to the subject, wherein the subject is not contemporaneously administered steroids.

3. A method of increasing remyelination in a subject in need thereof, the method comprising administering a repository corticotropin injection to the subject in an amount sufficient to show increased remyelination in a subsequent diagnostic test.

4. The method of claim 1, wherein the subject has been diagnosed with a demyelinating disorder of the CNS.

5. The method of claim 1, wherein the subject has been diagnosed with a leukodystrophic demyelinating disorder of the CNS.

6. The method of claim 1, wherein the subject has been diagnosed with a myelinoclastic demyelinating disorder of the CNS.

7. The method of claim 1, wherein the subject has been diagnosed with a demyelinating disorder of the peripheral nervous system.

8. The method of claim 1, wherein the subject has been diagnosed with a demyelinating disorder of the optic nerve.

9. The method of claim 1, wherein the subject has been diagnosed with a demyelinating disorder of the CNS that is not multiple sclerosis (MS).

10. The method of claim 1, wherein the subject has been diagnosed with a demyelinating disorder of the CNS that is not ALS.

11. The method of claim 1, wherein the subject has been diagnosed with a demyelinating disorder of the CNS that is not ALS or MS.

12. The method of claim 1, wherein the repository corticotropin injection is Acthar gel.


SEQUENCE LISTING (1 sequence)

SEQ ID NO: 1 - protein, 39 aa, Porcine
SYSMEHFRWGKPVGKKRRPVKVYPDGAEDQLAEAFPLEF